(19) United States
(12) Patent Application Publication
(10) Pub. No.: US 2024/0050340 A1
(43) Pub. Date: Feb. 15, 2024

(54) COMPOSITIONS AND METHODS FOR TREATING KERATIN FIBERS

(71) Applicant: L'Oreal, Paris (FR)

(72) Inventors: Cho-Cho KHINE, Scotch Plains, NJ (US); Ronak RUGHANI, Edison, NJ (US)

(21) Appl. No.: 18/231,735

(22) Filed: Aug. 8, 2023

Related U.S. Application Data
(60) Provisional application No. 63/396,186, filed on Aug. 8, 2022.

(30) Foreign Application Priority Data
Nov. 4, 2022 (FR) 2211494

(51) Int. Cl. A61K 8/362; A61Q 5/00; A61Q 5/06

(52) U.S. Cl. CPC A61K 8/362; A61Q 5/002; A61Q 5/06; A61K 2800/48

(57) ABSTRACT

The disclosure relates to methods of treating curly hair in connection with a bleaching process. The methods can improve and/or restore hair integrity, curl definition, and/or durability; prevent or reduce curl elongation; improve frizz and/or volume control; and/or prevent or reduce damage to the hair related to the bleaching process. The methods comprise applying a composition to the hair before and/or after the hair is treated with a bleaching composition, where the composition comprises (a) at least one carboxylic acid or salt thereof; (b) at least one cyclodextrin or derivative thereof; (c) at least one polysaccharide; (d) at least one polyol; and (e) water.

### CROSS REFERENCE TO RELATED APPLICATIONS

This application claims the benefit of priority to U.S. Provisional Application No. 63/396,186, filed on Aug. 8, 2022, and FR Application No. 2211494, filed Nov. 4, 2022, both of which are incorporated by reference herein in their entireties.

### TECHNICAL FIELD

The present disclosure relates to compositions and methods for treating curly or wavy hair in a bleach routine for maintaining and/or preventing curl elongation, maintaining and/or improving curl definition, and maintaining and/or restoring hair integrity, as well as providing additional overall sensorial benefits such as frizz and/or volume control.

### BACKGROUND

Consumers with curly hair may desire to maintain the curls and may also desire to gain better curl definition and/or regularity, and to improve curl retention. However, conventional color/bleach processes or routines often cause curl loosening and/or shrinkage and result in unwanted elongation of hair, as well as distortion of curl shape. In addition, a convention color/bleach process may increase frizz and porosity to the hair, cause extreme fragile fiber damage, and result in poor fixation and reflection of the hair. As such, curly hair consumers often see hair color/bleach as a curl enemy.

Although products exist that are meant to reduce frizziness and/or improve curl definition, these products have drawbacks such as imparting a greasy feel and/or a crunchy feel to the hair, weighing the hair down, or being ineffective, particularly in humid climates. In addition, currently available leading bleach products do not have a solution to fix both problems of curl loosening/shrinkage and damage.

Increasingly, consumers also seek hair products that provide a natural look and feel and a light-weight feel, while imparting longer-lasting styling or shaping benefits to hair. Further, consumers seek products that offer multiple benefits, for example, combining frizz reduction and style hold with smoothness, maintaining or preventing curl elongation or lengthening while providing good curl definition, to hair during bleach routines. Moreover, consumers desire hair products that can protect hair from external factors such as high humidity that causes the hair to become very frizzy, unmanageable, and lose its shape and style, or factors such as mechanical or physical or other external stresses on hair, for example, external stresses on hair during the process of bleaching hair.

As such, there is a need for new and improved products and methods that can improve the definition of the curls, maintain curls or prevent curl elongation, and/or provide frizz and/or volume control to the hair, as well as enhance the overall integrity and visual appearance of curly hair subjected to bleach.

The present disclosure addresses this need and aims at developing new compositions and methods using the disclosed compositions that can deliver one or more lasting caring and styling/shaping benefits described above to hair subjected to bleach. The present disclosure further aims at developing new bleach routines that can prevent curl elongation, maintain and/or improve hair integrity, and reduce damage to the hair.

### SUMMARY

The present disclosure relates to methods of using compositions according to the present disclosure to treat keratin fibers, such as curly or wavy hair, subjected to bleach for preventing or reducing curl elongation, improving fiber integrity, curl definition, and durability, providing frizz and/or volume control, providing smoothness, as well as avoiding or reducing damage to the hair. The present disclosure also relates to bleach processes or routines for bleaching curly hair while at the same time preventing or restoring hair integrity, preventing curl elongation, and/or maintaining or improving curl definition and hair shape.

It has now surprisingly been discovered that a combination of components according to the disclosure, particularly when applied to curly hair before or after treating the hair with a hair bleaching composition, can maintain or improve fiber integrity, prevent or maintain curl elongation and curl loosing due to the bleach process, and at the same time provide additional sensorial benefits such as frizz and/or volume control, smoothness, natural feel hydration, and light weight feel to the hair. These benefits have been shown to persist, even in conditions of high humidity, and last even after the treated hair is washed with or without shampoos.

In various embodiments, the methods for treating curly hair according to the present disclosure comprise applying to the curly hair a hair treatment composition; and optionally, rinsing off the composition, wherein the treatment composition is applied to the hair before, during, or after the bleaching composition is applied to the hair. The hair treatment composition comprises (a) at least one carboxylic acid or salt thereof; (b) at least one cyclodextrin or derivative thereof; (c) at least one polysaccharide; (d) at least one polyol; and (e) water; wherein the pH of the hair treatment composition is less than 7. The method is typically for maintaining or enhancing curl definitions and/or durability, maintaining or preventing curl elongation, improving and/or restoring hair integrity, and/or providing frizz and/or volume control to the hair.

The present disclosure further relates to methods of bleaching hair or a process of treating and bleaching hair, particularly curly hair.

In various embodiments, the process disclosed herein is a pre-treatment bleach routine or process comprising (1) applying a treatment composition to the hair and allowing the treatment composition to stay on the hair for a desired period of time of about 1 minute to about 30 minutes, preferably, about 3 minutes to about 10 minutes, or about 5 minutes; wherein the treatment composition comprises: (a) at least one carboxylic acid or salt thereof; (b) at least one cyclodextrin or derivative thereof; (c) at least one polysaccharide; (d) at least one polyol; and (e) water; wherein the pH of the composition is less than 7; (2) after the treatment composition is allowed to stay on the hair for the desired period of time, applying a bleaching composition to the hair while the treatment composition is left on the hair; (3) subsequently rinsing off the treatment composition and the bleaching composition from the hair after the bleaching composition is allowed to stay on the hair for a desired period of time ranging from about 20 minutes to about 1 hour; (4) shampooing the hair one or more times after rinsing the treatment composition and the bleaching composition off; (5) subsequently allowing the hair to air dry or drying the hair with a hair dryer; and (6) optionally, repeating steps of (1)-(5).

In various embodiments, the process disclosed herein is a post-treatment bleach routine or process comprising (1) applying a bleaching composition to the hair; (2) subsequently rinsing off the bleaching composition from the hair after the bleaching composition is allowed to stay on the hair for a desired period of time ranging from about 20 minutes to about 1 hour; (3) optionally, shampooing the hair twice after rinsing off the bleaching composition; (4) after rinsing off the bleaching composition, or after shampooing the hair if shampooing is present, applying to the hair a treatment composition comprising: (a) at least one carboxylic acid or salt thereof; (b) at least one cyclodextrin or derivative thereof; (c) at least one polysaccharide thickening agent; (d) at least one polyol; and (e) water; wherein the pH of the treatment composition is less than 7; (5) optionally, rinsing off the treatment composition from the hair; (6) subsequently allowing the hair to air dry or drying the hair with a hair dryer; and (7) optionally, repeating steps of (1)-(6).

In various embodiments, the process disclosed herein is a mix-treatment bleach routine or process comprising applying a mixture of a treatment composition and a bleaching composition to the hair; rinsing the mixture from the hair after the mixture is allowed to remain on the hair for about 20 minutes to about 1 hour; washing the hair with a shampoo one or more times after the mixture is rinsed off; and allowing the hair to air dry or drying the hair with a hair dryer; wherein the treatment composition comprises (a) at least one carboxylic acid or salt thereof; (b) at least one cyclodextrin or derivative thereof; (c) at least one polysaccharide; (d) at least one polyol; and (e) water; wherein the pH of the composition is less than 7.

### BRIEF DESCRIPTION OF THE FIGURES

The accompanying drawings, which are incorporated herein and constitute part of this specification, illustrate exemplary embodiments of the disclosure, and, together with the general description given above and the description provided herein, serve to explain features of the disclosure.

FIG. 1 is a set of schematic diagrams illustrating an exemplary pre-treatment bleach routine (R1), mix-treatment bleach routine (R2), and post-treatment bleach routine (R3) according to the disclosure.

FIG. 2 is a set of photos showing Caucasian curly hair swatches treated with inventive composition 1A and comparative compositions C1 and C2 in an exemplary pre-treatment bleach routine (R1), mix-treatment bleach routine (R2), and post-treatment bleach routine (R3) according to the disclosure, compared to the untreated hair and hair treated with a bleaching composition alone.

FIG. 3 is a graph showing break stress of the Caucasian curly hair swatches respectively treated with compositions 1A, C1, and C2 in an exemplary pre-treatment bleach routine, compared to untreated hair swatch and hair treated with a bleaching composition alone.

FIG. 4 is a graph showing cycles to break (CTB) of hair swatches respectively treated with compositions 1A, C1, and C2 in an exemplary pre-treatment bleach routine compared to the hair treated with the bleaching composition alone.

FIG. 5 is a graph showing the break stress (wet MTT) of Caucasian curly hair swatches treated with composition 1A respectively in an exemplary pre-treatment bleach routine (R1), mix-treatment bleach routine (R2), and post-treatment bleach routine (R3), compared to untreated hair and hair bleached twice (2×) alone.

FIG. 6 is a graph showing the fiber durability (cycles to break (CTB)) of the Caucasian curly hair swatches treated with composition 1A respectively in an exemplary pre-treatment bleach routine (R1), mix-treatment bleach routine (R2), and post-treatment bleach routine (R3), compared to hair bleached twice (2×) alone.

FIG. 7 is a graph showing the denaturation temperature of the Caucasian curly hair swatches treated with composition 1A respectively in an exemplary pre-treatment bleach routine (R1), mix-treatment bleach routine (R2), and post-treatment bleach routine (R3) compared to untreated hair and hair treated with the bleaching composition alone.

FIG. 8 shows the RUMBA 2D images of the Caucasian curly hair swatches treated and bleached according to an exemplary pre-treatment bleach routine (R1), mix-treatment bleach routine (R2), and post-treatment bleach routine (R3), compared to untreated hair and hair bleached twice (2×) alone.

FIG. 9 shows African-American hair swatches having tight curls treated with the bleaching composition once (1×) and twice (2×) alone, and hair swatches treated with composition 1A and the bleaching composition according to the pre-treatment bleach routine once (1A(pre)+bleached 1×) and twice (1A(pre)+bleached 2×), where the hair was pre-treated with composition 1A before the application of the bleaching composition, compared with an untreated hair swatch.

FIG. 10 is a graph showing the fiber durability (cycles to break (CTB)) of Caucasian curly hair, swatches (CP4) and African-American hair swatches (CP7) treated with composition 1A in the pre-treatment bleach routine disclosed herein, compared to the cycles to break of the untreated and bleached alone Caucasian and the African-American hair swatches.

It is to be understood that the foregoing and following descriptions are exemplary and explanatory only, and are not intended to be restrictive of any subject matter claimed.

### DETAILED DESCRIPTION

The disclosure relates to compositions and methods of using the compositions for treating keratin fibers, such as curly or wavy hair, in bleach routines for improving or restoring hair integrity, curl definitions, and/or durability; preventing or reducing curl elongation; improving frizz and/or volume control; providing shape control and/or discipline; and/or preventing or reducing damage to the hair subjected to bleach. The methods comprise applying a treatment composition according to the disclosure to hair (particularly) before or after the hair is bleached. The treatment composition comprises (a) at least one carboxylic acid and/or salt thereof; (b) at least one cyclodextrin and/or derivative thereof; (c) at least one polysaccharide; (d) at least one polyol; and (e) water. The pH of the treatment composition is less than 7.

The present disclosure further relates to hair bleach routines or processes for treating and bleaching hair, particularly curly hair. The bleach routines or processes include applying a treatment composition disclosed herein to the hair, and applying a bleaching composition to the hair, where the treatment composition is applied to the hair before or after the bleaching composition is applied to the hair.

I. Compositions

In various embodiments, compositions according to the disclosure comprise (a) at least one carboxylic acid and/or salt thereof; (b) at least one cyclodextrin and/or derivative thereof; (c) at least one polysaccharide thickening agent; (d) at least one polyol; and (c) water. The pH of the composition is less than 7. The compositions, when used in a bleach routine for curly hair, can prevent or reduce curl elongation, prevent or restore hair integrity, and/or provide lasting curl definition, frizz and/or volume control, as well as overall sensorial properties to the hair.

### Carboxylic Acids or Salts Thereof

Compositions according to the disclosure comprise at least one carboxylic acid or salt thereof. Mixtures of carboxylic acids and/or salts of carboxylic acids may be chosen. As used herein, the term “carboxylic acid” includes salts of carboxylic acids whether or not expressly stated. The carboxylic acids which may be used include, in various embodiments, mono-carboxylic acids (one carboxylic group) or poly-carboxylic acids (two or more carboxylic groups). Preferably, the carboxylic acids are non-polymeric carboxylic acids. The carboxylic acids may be chosen, in certain embodiments, from those having a molecular weight of less than about 900 g/mol, such as less than about 700 g/mol, less than about 500 g/mol, less than about 300 g/mol, or less than about 200 g/mol.

In some embodiments, the at least one carboxylic acid may be chosen from mono-carboxylic acids such as formic acid, acetic acid, propionic acid, butyric acid, valeric acid, caproic acid, entanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, lactic acid, or salts thereof.

In further embodiments, the at least one carboxylic acid may be chosen from dicarboxylic acids such as oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, or salts thereof.

In yet further embodiments, the at least one carboxylic acid may be chosen from tri-carboxylic acids such as citric acid, isocitric acid, aconitric acid, propane-1,2,3-tricarboxylic acid, benzene-1,3,5-tricarboxylic acid, or salts thereof.

In still further embodiments, the at least one carboxylic acid may be chosen from poly-carboxylic acids that have more than three carboxylic groups, such as phytic acid.

In various embodiments, the compositions comprise a mixture of two or more carboxylic acids and/or salts thereof. For example, the compositions may comprise a mixture of two or more mono-carboxylic acids and/or salts thereof, or may comprise a mixture or two or more poly-carboxylic acids and/or salts thereof, such as two or more di-carboxylic acids, two or more tri-carboxylic acids, or a mixture of one or more di-carboxylic acids and one or more tri-carboxylic acids, and so on. Mixtures of one or more mono-carboxylic or salts thereof with one or more poly-carboxylic acids or salts thereof are also contemplated. In further embodiments, the composition comprises only one carboxylic acid or a salt thereof.

In certain embodiments, the at least one carboxylic acid is chosen from citric acid, lactic acid, malic acid, maleic acid, tartaric acid, glycolic acid, phytic acid, salts thereof, or a mixture of two or more of the foregoing.

In various embodiments, the compositions comprise a total amount of carboxylic acids and/or salts thereof ranging from about 0.01% to about 20% by weight, such as about 0.1% to about 18%, about 0.5% to about 15%, or about 1% to about 13% by weight, relative to the total weight of the composition. By way of non-limiting example, the compositions may comprise a total amount of carboxylic acids and/or salt thereof ranging from about 0.5% to about 15%, by weight, for example, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 2% to about 15%, from about 2% to about 12%, from about 2% to about 10%, from about 2% to about 9%, from about 2% to about 8%, from about 2% to about 7%, from about 2% to about 6%, or from about 2% to about 5%, by weight, relative to the total weight of the composition, including all ranges and subranges thereof. In some embodiments, the compositions comprise a total amount of carboxylic acids and/or salts thereof ranging from about 0.5% to about 13%, such as from about 0.5% to about 10%, or from about 0.5% to about 8% by weight, relative to the total weight of the composition.

In some embodiments, the at least one carboxylic acid comprises or consists of citric acid, lactic acid, malic acid, maleic acid, tartaric acid, glycolic acid, phytic acid, salts thereof, or a mixture thereof, in an amount ranging from about 0.1% to about 10%, such as from about 0.3% to about 9%, or from about 0.5% to about 8% by weight, relative to the total weight of the composition, including all ranges and subranges thereof.

In still further embodiments, the composition comprises citric acid and/or salts thereof in an amount ranging from about 1% to about 5%, such as from about 1% to about 4%, from about 1% to about 3%, from about 1.5% to about 4%, from about 1.5% to about 3.5%, from about 1.5% to about 3%, from about 2% to about 4%, from about 2% to about 3.5%, from about 2% to about 3%, or from about 2.25% to about 2.75%, including all ranges and subranges thereof, or is about 1%, about 1.5%, about 2%, about 2.5%, about 3%, or about 3.5% by weight, relative to the total weight of the composition, including all ranges and subranges using any of the foregoing as upper or lower limits.

### Cyclodextrins or Derivatives Thereof

Compositions according to the disclosure comprise at least one cyclodextrin or derivative thereof. As used herein, the term “cyclodextrins” includes derivatives, whether or not expressly stated. Cyclodextrins are a family of cyclic oligosaccharides consisting of a macrocyclic ring of glucose subunits joined by α-1,4 glycosidic bonds.

The cyclodextrins that can be used include those of the formula:

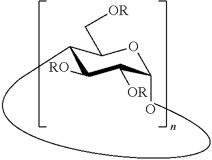

wherein:
R is chosen from H, CH₃, or a hydroxypropyl group, and
n ranges from 6-8.

For example, in embodiments where R=H, the cyclodextrin may be α-cyclodextrin (n=6), β-cyclodextrin (n=7), or γ-cyclodextrin (n=8). By way of example, α-cyclodextrin sold by the company WACKER under the name CAVAMAX W6 PHARMA, β-cyclodextrin sold by the company WACKER under the name CAVAMAX W7 PHARMA, or γ-cyclodextrin sold by the company WACKER under the name CAVAMAX W8 PHARMA can be used.

In other embodiments where R=CH₃, the cyclodextrin may be a methyl-cyclodextrin, such as methyl-α-cyclodextrin (n=6), methyl-β-cyclodextrin (n=7), or methyl-γ-cyclodextrin (n=8). For example, the methyl-β-cyclodextrin sold by the company WACKER under the name CAVASOL W7 may be chosen.

In various embodiments, the at least one cyclodextrin may comprise a mixture of cyclodextrins and/or derivatives thereof. For example, the at least one cyclodexctrin may be a mixture of α-cyclodextrin, β-cyclodextrin, and/or γ-cyclodextrin. In another embodiment, the at least one cyclodextrin includes β-cyclodextrin. In yet a further embodiment, the cyclodextrin is only β-cyclodextrin, and no other cyclodextrins or derivatives thereof are present in the composition.

In various embodiments, the compositions comprise a total amount of cyclodextrins and/or derivatives thereof in an amount ranging from about 0.01% to about 20% by weight, including all ranges and subranges thereof, such as from about 0.1% to about 18%, from about 0.5% to about 18%, from about 0.5% to about 15%, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 18%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, or from about 1% to about 3% by weight, relative to the total weight of the composition. In some embodiments, the compositions comprise a total amount of cyclodextrins and/or derivatives thereof ranging from about 1% to about 20%, such as from about 0.1% to about 18%, from about 0.5% to about 15%, from about 0.5% to about 10%, from about 0.5% to about 8%, from about 1% to about 13%, from about 1% to about 7%, or from about 1% to about 5% by weight, relative to the total weight of the composition.

In some embodiments, the compositions comprises β-cyclodextrin in an amount ranging from about 0.1% to about 10%, such as from 0.2% to about 8%, from about 0.3% to about 7%, from about 0.4% to about 6%, from about 1% to about 10%, from about 1% to about 8%, from about 1% to about 5%, from about 1% to about 3% by weight, relative to the total weight of the composition, including all ranges and subranges thereof.

In various embodiments, it is particularly advantageous to choose total amounts of carboxylic acids (including salts) and cyclodextrins (including derivatives) such that a mole ratio of carboxylic acid(s):cyclodextrin(s) ranges from about 0.5:1 to about 25:3, for example about 4:1 to about 15:1, about 5:1 to about 12:1, or about 6:1 to about 10:1, such as, for example, about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 3:2, about 5:2, about 7:2, about 9:2, about 11:2, about 13:2, about 15:2, about 17:2, about 19:2, about 1:3, about 2:3, about 4:3, about 5:1, about 7:3, about 8:3, about 10:3, about 11:3, about 13:3, about 14:3, about 16:3, about 17:3, about 19:3, or about 20:3, including all ranges and subranges using any of the foregoing as upper or lower limits.

In some embodiments, compositions according to the disclosure may have a weight ratio of (a) the total amount of carboxylic acids and salts thereof to (b) the total amount of cyclodextrins and derivatives thereof ranging from about 1:3 to about 6:1, for example from about 1:3 to about 3:1, from about 1:2.5 to about 2.5:1, from about 1:2 to about 2:1, from about 1:1 to about 1.75:1, from about 1.5:3 to about 3:1.5, from about 1.5:2.5 to about 2.5:1.5, or from about 1.5:2 to about 2:1.5, or may be about 1:3, about 1:2.5, about 1:2, about 1:1.5, about 1:1, about 1.5:1, about 2:1, about 2.5:1, about 3:1, about 3.5:1, about 4:1, about 4.5:1, about 5:1, about 5.5:1, about 6:1, about 1.5:3, about 1.5:2.5, about 1.5:2, about 2:1.1, about 2.5:1.5, about 3:1.5, about 3.5:1.5, about 4:1.5, about 4.5:1.5, about 5:1.5, about 5.5:1.5, or about 6:1.5, including all ranges and subranges using any of the foregoing as upper or lower limits.

### Polysaccharide Thickening Agents

Compositions according to the disclosure comprise at least one polysaccharide as a thickening agent. The term “thickening agent” may be referred to interchangeably to as “thickener” or “viscosity modifying agent.” The at least one polysaccharide agent may be chosen from anionic polysaccharide thickening agents, nonionic polysaccharide thickening agents, or mixtures of two or more thereof. Polysaccharide thickening agents that can be used include, by way of example only, inulins, celluloses, starches, guar gums, xanthan gums, pullulan gums, alginate gums, agar-agar gums, carrageenan gums, gellan gums, gum arabics, xyloses, tragacanth gums, or derivatives of the foregoing. In further embodiments, cellobiose, maltodextrin, scleroglucan, chitosan, ulvan, fucoidan, alginate, pectin, or heparin. In some embodiments, mixtures of two or more of any of these may also be chosen.

Non-limiting examples of gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, dehydroxanthan gum, and biosacharide gum. Modified gums or derivatives of gums may also be used, such as, for example, deacylated gellan gum, welan gum, or hydroxypropylated guar gum, such as Jaguar HP 105 sold by Rhodia.

In some embodiments, suitable polysaccharide thickening agents that can be used are chosen from gums, such as gum arabic, tragacanth gum, karaya gum, guar gum, gellan gum, tara gum, locust bean gum, tamarind gum, xanthan gum, dehydroxanthan gum, Seneca gum, sclerotium gum, gellan gum, and the like.

Non-limiting examples of celluloses include hydroxyalkylcelluloses, such as hydroxymethylcellulose, methylhydroxyethylcellulose, hydroxyethylcelluloses, hydroxypropylmethylcellulose, hydroxybutylcellulose, hydroxyethylmethylcellulose (also known as methyl hydroxyethylcellulose) or hydropropylcelluloses, which may or may not contain a fatty chain. One particularly suitable hydroxypropylmethylcellulose is Methocel F4M sold by Dow Chemicals (INCI name: hydroxypropylmethylcellulose). Celluloses modified with groups comprising one or more nonionic fatty chains that can be used include hydroxyethylcelluloses, preferably nonionic hydroxyethylcelluloses, modified by groups comprising at least one fatty chain, such as alkyl, arylalkyl or alkylaryl groups, or their mixtures, and in which the alkyl groups are preferably C8-C22 alkyl groups, such as the product NATROSOL™ Plus Grade 330 CS (C16 alkyls), sold by Aqualon, corresponding to the INCI name cetylhydroxyethylcellulose, or the product BERMOCOLL® EHM 100 sold by Berol Nobel, and those modified with alkylphenyl polyalkylene glycol ether groups, such as the product AMERCELL POLYMER®HM-1500 (nonylphenyl polyethylene glycol (15) ether) sold by Amerchol that corresponds to the INCI name nonoxynyl hydroxyethylcellulose. Other particular thickening agents suitable for use in the compositions described herein are hydroxypropyl guar gums.

Non-limiting examples of starches include modified starches, starch-based polymers, methylhydroxypropyl starch, potato starch, modified potato starch, wheat starch, rice starch, starch crosslinked with octenyl succinic anhydride, starch oxide, dialdehyde starch, dextrin, British gum, acetyl starch, starch phosphate, carboxymethyl starch, hydroxyethyl starch, and hydroxypropyl starch.

In some embodiments, the polysaccharide thickening agents are chosen from carrageenan, xanthan gum, cellulose, carboxymethylcellulose, hydroxyethylcellulose, hydroxypropylcellulose, hydroxypropylmethylcellulose, methylcellulose, starch, starch acetate, hydroxyethyl starch, hydroxypropyl starch, guar gum, carboxymethyl guar gum, carboxymethylhydroxypropylguar gum, hydroxyethyl guar gum, hydroxypropyl guar, xylose gum, cellobiose, dextrin, maltodextrin, scleroglucan, chitosan, ulvan, fucoidan, alginate, pectin, heparin, inulin, levan, or mixtures thereof. In further embodiments, the polysaccharide thickening agents are chosen from sclerotium gum, carrageenan, xanthan gum, cellulose gum, cyclodextrin, or mixtures of two or more thereof.

In various exemplary embodiments, the total amount of polysaccharide thickening agents may vary, and may typically range from about 0.05% to about 5%, such as from about 0.1% to about 5%, from about 0.1% to about 4%, from about 0.1% to about 3%, from about 0.1% to about 2%, from about 0.1% to about 1%, from about 0.5% to about 5%, from about 0.5% to about 4%, from about 0.5% to about 3%, from about 0.5% to about 2%, from about 0.5% to about 1%, from about 1% to about 5%, from about 1% to about 4%, from about 1% to about 3%, or from about 1% to about 2% by weight, relative to the total weight of the composition, including all ranges and subranges thereof. In some embodiments, the total amount of polysaccharide thickening agents may range from about 0.1% to about 5%, such as from about 0.25% to about 4%, from about 0.5% to about 3%, or from about 0.5% to about 2.5%, or may be about 1%, about 1.5%, about 2%, about 2.5%, about 3%, or about 3.5% by weight, relative to the total weight of the composition, including all ranges and subranges using any of the foregoing as upper or lower limits.

For example, the compositions may, in exemplary embodiments, comprise a total amount of polysaccharide thickening agents ranging from about 0.1% to about 5%, from about 0.5% to about 4%, or from about 0.75% to about 3%, preferably from about 0.5% to about 2% by weight, relative to the total weight of the composition, wherein the polysaccharide thickening agent comprises at least one polysaccharide thickening agent may be chosen from gum arabic, tragacanth gum, karaya gum, guar gum, gellan gum, tara gum, locust bean gum, tamarind gum, xanthan gum, dehydroxanthan gum, Seneca gum, sclerotium gum, gellan gum, etc. In various embodiments, the composition comprises xanthan gum in an amount ranging from about 0.1% to about 5%, such as from about 0.25% to about 4%, from about 0.5% to about 3%, or from about 0.5% to about 2.5%, or may be about 1%, about 1.5%, about 2%, about 2.5%, about 3%, or about 3.5% by weight, relative to the total weight of the composition, including all ranges and subranges using any of the foregoing as upper or lower limits.

### Polyols

Compositions according to the disclosure comprise at least one polyol. By way of example only, the at least one polyol may be chosen from C2-C16 polyols, such as C2-C12 or C2-C8 polyols. The at least one polyol may optionally be chosen from diols and triols. The at least one polyol may be linear or branched, saturated or unsaturated, and substituted or unsubstituted. Any stereoisomer of the polyols may also be used. Polyol(s) in the combination disclosed herein may act as conditioning or smoothing agents and improve overall sensorial properties of hair and enhance curl definition.

In various exemplary embodiments, the polyols may be chosen from glycols such as ethylene glycol, propylene glycol, butylene glycol, hexylene glycol, pentylene glycol, 1,3-propanediol, diethylene glycol, triethylene glycol, tetraethylene glycol, pentaethylene glycol, dipropylene glycol, caprylyl glycol, glycerin, diglycerin, ethylhexyglycerin, or mixtures of two or more thereof.

In other embodiments, the polyols may be chosen from, for example, propanediol, butanediol, pentanediol, hexanediol, heptanediol, octanediol, 1,2,6-hexanetriol, 1,2,4-butanetriol, trimethylolpropane, 2-butene-1,4-diol, 2-ethyl-1,3-hexanediol, 2-methyl-2,4-pentanediol, caprylyl glycol, 1,2-hexanediol, 1,2-pentanediol, 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, 2-ethyl-1,3-hexanediol, 4-methyl-1,2-pentanediol, or mixtures of two or more thereof.

In some embodiments, the compositions according to the disclosure comprise at least two polyols, for example at least two polyols chosen from diols and/or triols. As one non-limiting example, a composition according to the disclosure may comprise a combination of at least one glycerin compound and at least one additional glycol compound.

In various embodiments, the total amount of polyols can be about 1% or more, such as about 2% or more, about 3% or more, about 4% or more, about 5% or more, about 6% or more, for example up to about 10% by weight, based on the total weight of the composition. In some embodiments, the total amount of polyol is present in the composition with a total amount ranging from about 0.1% to about 10%, including all subranges therebetween, such as from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 8%, from about 0.5% to about 7%, from about 0.5% to about 6%, from about 0.5% to about 5%, from about 0.5% to about 4% by weight, from about 0.5% to about 3%, from about 0.5% to about 2%, or from about 0.5% to about 1.5% by weight, relative to the total weight of the composition. In some embodiments, the total amount of polyols ranges from about 0.1% to about 5%, such as from about 0.25% to about 4%, from about 0.5% to about 3%, or from about 0.5% to about 2.5%, or may be about 1%, about 1.5%, about 2%, about 2.5%, about 3%, or about 3.5% by weight, relative to the total weight of the composition, including all ranges and subranges using any of the foregoing as upper or lower limits.

In some exemplary and non-limiting embodiments, the compositions comprise at least one polyol chosen from diols and/or triols, and the total amount of polyols is about 1% or more, about 1.5% or more, about 2% or more, about 3% or more, about 4% or more, or about 5% or more by weight, for example up to about 10% by weight, relative to the total weight of the composition, and preferably comprises glycerin.

### Water

Compositions according to the disclosure comprise water. In various embodiments, the amount of water in a composition disclosed herein is generally greater than 50%, and may, for example, range from about 70% to about 98%, including all subranges therebetween, such as from about 75% to about 98%, from about 80% to about 98%, from about 85% to about 98%, from about 90% to about 98%, from about 95% to about 98%, from about 70% to about 95%, from about 75% to about 95%, from about 80% to 95%, from about 85% to 95% by weight, relative to the total weight of the composition.

### Additional Components

Compositions according to the disclosure may optionally comprise one or more additional components suitable for use in such compositions. Non-limiting examples of such additional components are provided below.

### pH Adjusters

Compositions disclosed herein may include one or more pH adjusters to increase or decrease the overall pH of the composition. For example, one or more acids may be included to decrease the pH of the cosmetic composition. Examples of suitable acids for decreasing the pH of the compositions may include one or more acids. The cosmetic composition may include one or more bases, such as sodium hydroxide, potassium hydroxide and the like, to increase the pH of the cosmetic composition. Additional or alternative acids and bases that are suitable for adjusting the pH of the compositions are readily known to one of ordinary skill in the art. For instance, in one embodiment, the pH adjuster is sodium hydroxide solution such as 0.1 M NaOH.

The amount of the pH adjuster in the compositions may be based on the desired pH of the final composition and/or product for improving curl definition and curl regularity, and/or preventing curl elongation. For example, the total amount of the pH adjuster may range from about 0.0001% to about 10% by weight, including ranges and sub-ranges therebetween, based on the total weight of the cosmetic composition.

### Preservatives

One or more preservatives may be included in the compositions described herein for treating hair. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, benzoic acid, chlorhexidine digluconate, ethylenediamine-tetraacetic acid (EDTA), potassium sorbate, and/or grapefruit seed extract, or a mixture thereof. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, Vitamin E (tocopherol), or mixtures thereof. In some cases, the hair-treatment compositions may include one or more preservatives selected from the group consisting of sodium benzoate, benzoic acid, chlorhexidine digluconate, chlorhexidine dihydrochloride, salicylic acid, phenoxyethanol, methyl paraben, or mixtures thereof.

The total amount of the one or more preservatives, when present, may vary. In some cases, the total amount of the one or more preservatives may range from about 0.01% to about 5%, from about 0.01% to about 4%, from about 0.01% to about 2%, from about or about 0.01% to about 3% by weight, relative to the total weight of the composition.

### Auxiliary Components

Compositions according to the disclosure may optionally comprise any auxiliary component suitable for use in such compositions. Such components may include, but are not limited to, dyes/pigments for adding color to the composition, moisturizing agents, fatty substances (e.g. oils, in particular polar oils such as plant and/or animal oils), thickeners other than those previously described, fillers, structuring agents, shine agents, antioxidants or penetrants, sequestrants, fragrances, buffers, dispersants, plant extracts, such as apricot seed powder, opacifiers, sunscreen agents, vitamins, and antistatic agents. Compositions according to the disclosure preferably do not include dyes/pigments for coloring the hair.

Optional auxiliary components may be present in an amount ranging up to about 15%, such as from about 0.001% to about 10%, from about 0.01% to about 5%, or from about 0.01% to about 1% by weight, relative to the total weight of the composition.

In various embodiments, the compositions are acidic and have a pH of less than 7. For example, the pH of the composition may range from about 1.5 to about 6.8, from about 2 to about 6.5, from about 2.5 to about 6, from about 3 to about 6.5, from about 3 to about 6, from about 3 to about 5.5, from about 3 to about 5, from about 3 to about 4, from about 3.5 to about 6.5, from about 3.5 to about 6, from about 3.5 to about 5.5, from about 3.5 to about 5, from about 3.5 to about 4.5, or from about 3.5 to about 4, including all ranges and subranges using any of the foregoing as upper or lower limits. In some exemplary embodiments, the pH of the composition ranges from about 3 to about 6.5. In further exemplary embodiments, the pH of the composition ranges from about 3 to about 4.

Compositions according to the disclosure may be rinse-off compositions or leave-in compositions, and are typically in the form of a gel, liquid, cream, or lotion, but are not so limited. In some embodiments, the compositions are a non-aerosol type product, which may be dispensed from a bottle, tube, or spray-bottle.

In some embodiments, the compositions may be a hair treatment product, a hair styling product, or a hair styling and managing product that can be used for styling hair while providing treatment and/or conditioning, as well as manageability benefits to the hair.

II. Methods

A. Methods of Making the Compositions

The disclosure also relates to methods of making compositions described herein. It may, in some embodiments, be particularly beneficial to choose specific mole or weight ratios of carboxylic acid(s) and/or salt(s), cyclodextrin(s) and/or derivative(s) thereof, and/or solvent(s) (e.g. deionized (DI) water). For example, by choosing specific mole or weight ratios of these components, it is possible to tailor the compositions so that greater or lesser degrees of curl definition and/or frizz control may be imparted.

Therefore, it may be useful to prepare a pre-mixture, or stock mixture, of concentrated combinations of carboxylic acid(s) and/or salt(s) thereof, cyclodextrin(s) and/or derivative(s) thereof, and solvent(s) with particular mole ratios or weight ratios thereof. In one embodiment, the pre-mixture comprises at least one carboxylic acid chosen from citric acid, and at least one cyclodextrin chosen from β-cyclodextrin.

In various embodiments, in the pre-mixture, carboxylic acids (including salts) and cyclodextrins (including derivatives) may be present in a mole ratio of carboxylic acid(s):cyclodextrin(s) ranging from about 0.5:1 to about 25:3, such as from about 4:1 to about 15:1, from about 5:1 to about 12:1, or from about 6:1 to about 10:1, or may be present in mole ratios of about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 3:2, about 5:2, about 7:2, about 9:2, about 11:2, about 13:2, about 15:2, about 17:2, about 19:2, about 1:3, about 2:3, about 4:3, about 5:1, about 7:3, about 8:3, about 10:3, about 11:3, about 13:3, about 14:3, about 16:3, about 17:3, about 19:3, or about 20:3, including all ranges and subranges using any of the foregoing as upper or lower limits. Correspondingly, the weight ratio of (a) the total amount of carboxylic acids and salts thereof to (b) the total amount of cyclodextrins and derivatives thereof ranging from about 1:3 to about 6:1, or may be present in weight ratios of about 1:3, about 1:2.5, about 1:2, about 1:1.5, about 1:1, about 1.5:1, about 2:1, about 2.5:1, about 3:1, about 3.5:1, about 4:1, about 4.5:1, about 5:1, about 5.5:1, or about 6:1, including all ranges and subranges using any of the foregoing as upper or lower limits.

Once the particular carboxylic acid(s), cyclodextrin(s), and solvent(s), as well as the desired mole and/or weight ratios thereof, are chosen, the pre-mixture may be prepared by any conventional method. As an illustratory example only, the pre-mixture may be prepared by simple mixing of the components at the chosen ratio, optionally with heating. For example, the mixture may be mixed at a temperature ranging from about 40° C. to about 100° C., such as about 50° C. to about 95° C. By way of example, the pre-mixture may be prepared by: first, mixing one or more carboxylic acids and/or salts thereof and one or more cyclodextrins and/or derivatives thereof in DI water; second, heating the mixture in a water bath at about 80° C. until the contents dissolve and the mixture forms a viscous liquid; third, leaving the mixture at about 80° C. for about 2 hours to allow the ingredients to complete the reaction, and; fourth, cooling the mixture to room temperature.

After the pre-mixture is prepared, it may then be diluted with additional solvent and other additives including polysaccharide(s) and polyol(s), e.g., xanthan gum and glycerin, to the desired concentration of the final composition according to the disclosure. The additional solvent may be the same as the solvent in the pre-mixture, may be different from the solvent in the pre-mixture, or may be a combination of the solvent in the pre-mixture with one or more solvents other than that in the pre-mixture.

In one exemplary and non-limiting embodiment, a composition according to the present disclosure comprises citric acid, beta-cyclodextrin, xanthan gum, and glycerin, may be prepared by diluting a pre-mixture of citric acid and beta-cyclodextrin to a desired concentration with additional solvent(s), e.g., DI water, adjusting the pH of the dilution with a pH adjuster, e.g., 0.1 M sodium hydroxide solution, subsequentially adding desired amounts of xanthan gum and glycerin to the dilution to obtain a mixture, and further mixing the mixture using a mixer or vortexer (or vortex mixer) to arrive at the compositions disclosed herein.

In various embodiments, compositions according to the disclosure may contain a weight ratio of pre-mixture:additional components ranging from about 1:99 to about 50:50. For example, compositions according to the disclosure may contain a weight ratio of pre-mixture:additional components ranging from about 2:98 to about 40:60 or about 3:97 to about 35:65, for example may be about 2.5:97.5, about 5:95, about 7.5:92.5, about 10:90, about 12.5:87.5, about 15:85, about 17.5:81.5, about 20:80, about 25:75, or about 30:70, including all ranges and subranges using any of the foregoing as upper or lower limits. In some exemplary embodiments, the weight ratio of pre-mixture:additional components ranges from about 5:95 to about 15:85.

B. Methods of Treating Hair

The disclosure also relates to methods of treating curly hair subjected to bleach processes in order to enhance or restore hair integrity, curl definition and/or durability; prevent or reduce curl elongation; improve frizz and/or volume control; and/or prevent or reduce damage to the hair, while lifting the color of the hair or lightening the hair. As used herein, the term “bleach routine” refers to a procedure or a sequence of actions including applying a bleaching composition and, optionally, one or more treatment compositions other than the bleaching composition to the hair for removing the color from the hair (hair lightening or lifting).

In various embodiments, the methods of treating hair according to the present disclosure generally comprise applying any of the compositions disclosed herein to the hair, particularly curly or wavy hair in a bleach routine. The degree of curliness or curl type of the hair may vary and is not limited. For example, the curls of hair been treated by the compositions disclosed herein may range from slightly wavy to tight curls, and may have different textures and colors. The curls of the hair may be natural or unnatural, such as obtained via a permanent hair styling process using chemicals that structurally change the texture of the hair. It is preferred that the compositions disclosed herein are applied to the hair before or after the hair is applied with a bleaching composition, instead of applying the compositions together with the bleaching composition, either in a mixture or applied on the hair at the same time.

In some embodiments, methods according to the disclosure may include applying an effective amount of a composition disclosed herein to hair having curls, where the hair is dry, wet, damp, or moist, before or after the hair is treated with a bleach composition, optionally rinsed with water and/or washed with a shampoo. As used herein, the term “effective amount” refers to an amount sufficient to provide a desired effect to the hair, taking into account the degree of curliness, the length, the volume, and the texture of the hair. In general, from about 0.1 grams to about 5 grams of product is applied per gram of hair, depending on the specific product formulation, hair length, hair volume, and hair style type.

The composition may be applied to the hair before or after the hair is shampooed and/or conditioned, or after the hair is rinsed with water. In some embodiments, the composition may be applied to the hair without shampooing or conditioning the hair before or after.

After the composition is applied on the hair, the hair may be massaged or combed through with fingers or by a means such as a comb or the like such that the composition is evenly distributed on the hair. In some embodiments, the treatment composition is allowed to stay on the hair for a desired period of time of no less than 1 minute, such as about 3 minutes to about 10 minutes, or about 5 minutes.

In some embodiments, the process is a pre-treatment process, where a treatment composition disclosed herein is applied to the hair and distributed evenly on the hair before the bleaching composition is applied to the hair. Before applying the treatment composition, the hair may be rinsed with water or shampooed. After the treatment composition is allowed to remain on the hair for a desired period of time as described above, the bleaching composition is then applied to the hair while the treatment composition remains on the hair. In some embodiments, the bleach composition may be rinsed from the hair after the bleach composition is allowed to remain on the hair for about 20 minutes to about 1 hour. After rinsing off the bleach composition, the hair may be further optionally washed with a shampoo at least once. Subsequently, the hair may be air dried or dried with a hair dryer.

In some embodiments, the process is a post-treatment process, where the compositions disclosed herein are applied to the hair after the hair is treated with a bleaching composition for a desired period of time such as from about 20 minutes to about 1 hour. The composition disclosed herein may be applied to the hair after rinsing the bleach composition from the hair and optionally washing the hair with a shampoo thereafter. The treatment composition may be massaged or allowed to stay on the hair for a period of time of no less than 1 minute, such as about 3 minutes to about 10 minutes, or about 5 minutes. Subsequently, the treatment composition may be optionally rinsed off. In some embodiments, the treated hair herein may be styled, e.g. scrunched, twisted, or the like, which may aid in defining the curls. In various embodiments, the treated hair may be air dried or dried by a dryer, for example a hood dryer or a blow dryer, where the composition may be rinsed from the hair before drying, or may be or allowed to remain on the hair as a leave-in product for any period of time as desired, for example, a few hours, overnight, or a few days, or until the next washing or rinsing of the hair. After the treated hair is dried, the hair may be passed by fingers to break the film formed on the hair by the ingredients in the components. Generally, in a post-treatment routine, when the composition disclosed herein is rinsed from the treated hair, the hair looks less stiff and not crunchy.

The present disclosure further relates to methods for bleaching hair, or hair bleach routines or processes, or a process for lifting the color of curly hair while providing benefits such as improving or restoring hair integrity, curl definitions, and/or durability; preventing or reducing curl elongation; improving frizz and/or volume control; and/or preventing or reducing damage to the hair. The bleach process or routine generally includes applying a treatment composition disclosed herein to the hair and applying a bleaching composition to the hair, where the treatment composition may be applied to the hair before or after the application of the bleaching composition.

In some embodiments, the bleach routine is a pre-treatment bleach routine, which comprises 1) applying a treatment composition disclosed herein to the hair and allowing the treatment composition to stay on the hair for a desired period of time of no less than 1 minute, such as about 1 minute to about 30 minutes, about 3 minutes to about 10 minutes, or about 5 minutes; 2) after the treatment composition is allowed to stay on the hair for the desired period of time of no less than 1 minute, applying a bleaching composition to the hair while the treatment composition is left on the hair; 3) subsequently rinsing off the treatment composition and the bleaching composition from the hair after the bleaching composition is allowed to stay on the hair for a desired period of time ranging from about 20 minutes to about 1 hour; 4) optionally shampooing the hair one or more times after rinsing the treatment composition and the bleaching composition off; and 5) subsequently allowing the hair to air dry or drying the hair with a hair dryer; and 6) optionally, repeating steps of (1)-(5).

In some embodiments, the bleach routine is a post-treatment routine, which comprises 1) applying a bleaching composition to the hair; 2) subsequently rinsing off the bleaching composition from the hair after the bleaching composition is allowed to stay on the hair for a desired period of time ranging from about 20 minutes to about 1 hour; 3) optionally, shampooing the hair twice after rinsing off the bleaching composition; 4) after rinsing off the bleaching composition, or after shampooing the hair if shampooing is present, applying to the hair a treatment composition disclosed herein; 5) optionally, rinsing off the treatment composition from the hair; 6) subsequently allowing the hair to air dry or drying the hair with a hair dryer; and 7) optionally, repeating steps of (1)-(6). Before the hair is dried in a final step, the hair may be styled, such as by scrunching the hair using hands.

It is to be understood that a pre-treatment or post-treatment process as described herein generally contemplates application of a composition according to the disclosure at a time that is relatively proximate to the bleaching process. For example, the pre-treatment routine may include application of a composition according to the disclosure within about 48 hours or less, such as about 24 hours or less, about 12 hours or less, about 6 hours or less, about 3 hours or less, about 2 hours or less, about 1 hour or less, or about 30 minutes or less before the bleaching process commences, or may occur substantially immediately prior to commencement of the bleaching process. Similarly, the post-treatment routine may include application of a composition according to the disclosure within about 48 hours after, such as no more than about 24 hours after, no more than about 12 hours after, no more than about 6 hours after, no more than about 3 hours after, no more than about 2 hours after, no more than about 1 hour after, or no more than about 30 minutes after the bleaching process concludes, or may occur substantially immediately after the bleaching process concludes.

In some embodiments, the bleach routine is a mix-treatment routine, which comprises applying a mixture of a treatment composition according to the present disclosure and a bleaching composition to the hair; rinsing the mixture from the hair after the mixture is allowed to remain on the hair for about 20 minutes to about 1 hour; washing the hair with a shampoo one or more times after the mixture is rinsed off; and allowing the hair to air dry or drying the hair with a hair dryer.

In some embodiments, a composition disclosed herein is used alone on the hair, without the use of other hair treatment compositions. In some other embodiments, after treating curly hair according to a pre-treatment bleach routine or post-treatment bleach routine, another hair treatment or styling composition, such as styling gel, hairspray, etc., may be applied to the hair. As such, a composition disclosed herein may be applied to the hair and layered with another composition that is also applied to the hair.

In various embodiments, the methods of treating hair with the compositions according to the disclosure, impart various long-lasting benefits described above to the hair, without a greasy feel or flaking, relative to hair not having been treated with a composition according to the disclosure. The term “long-lasting,” as used herein, means that the curl defining, prevention of elongation, volume control, frizz control, hair integrity effects and other benefits imparted to the hair may remain a desired length of time, such as at least one or a few hours, a few days, even after the hair is rinsed or washed.

III. Kits

The disclosure further relates to kits comprising a first container containing a composition according to the present disclosure and/or a concentrated pre-mixture as disclosed herein, and one or more additional containers containing one or more hair care/treatment compositions other than the compositions disclosed herein, such as, for example, a shampoo, a conditioner, a hair bleach composition, an oxidizing composition, a hair mask, or a hair styling composition.

The pre-mixture, or stock mixture, is a concentrated combination of a) at least one carboxylic acid or salt thereof; (b) at least one cyclodextrin or derivative thereof; (c) at least one polysaccharide thickening agent; and (d) at least one polyol. The pre-mixture may be substantially free of water. For example, the pre-mixture may have less than about 20%, less than about 18%, less than about 15%, less than about 12%, less than about 10%, less than about 8%, less than about 5%, less than about 3%, less than about 2%, or less than about 1% water. The pre-mixture can then be diluted, e.g. with water, to result in a composition according to the present disclosure near or at the time of use. The kit may optionally further comprise instructions instructing a user regarding appropriate mixing steps, ratios, timing, etc. when a pre-mixture is included, or instructions for using the compositions, including various treatment routines disclosed herein. The kits in various embodiments may comprise at least one container suitable for containing and/or dispensing the compositions described herein for applying to the hair. In some other embodiments, a kit may comprise a first container containing a composition according to the disclosure, and at least one additional container comprising a composition according to the disclosure or a composition not according to the disclosure, such as, for example, a shampoo, a conditioner, a hair mask, or a hair styling composition.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, or a salt of F may be included, or a mixture of any two of A, B, C, D, E, F, a salt of A, a salt of B, a salt of C, a salt of D, a salt of E, and a salt of F may be included.

The term “and/or” should be understood to include both the conjunctive and the disjunctive. For example, “citric acid and/or salts thereof” means “citric acid and salts thereof” as well as “citric acid or salts thereof,” and expressly covers instances of either.

As used herein, the phrases “and mixtures thereof,” “and mixtures of two or more thereof,” “and a mixture thereof,” “and combinations thereof,” “and a combination thereof,” “or mixtures thereof,” “or a mixture thereof,” “or combinations thereof,” “or combinations of two or more thereof,” “or a combination thereof,” and the like are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts given herein are relative to the amount of active material, unless otherwise indicated.

All percentages, parts and ratios herein are based upon the total weight of the compositions of the present disclosure, unless otherwise indicated.

As used herein, the terms “applying a composition onto keratin fibers” and “applying a composition onto hair” and variations of these phrases are intended to mean contacting the keratin materials including hair, with at least one of the compositions of the disclosure, in any manner. It may also mean contacting the keratin fibers in an effective amount.

As used herein, the term “curly hair” refers to any hair including a curl. The curl may be natural or unnatural, i.e., formed by chemical treatment or physical treatment of the hair. The degree of curliness of the hair may vary and is not limited.

As used herein, hair with improved or enhanced curl definition may have curls with a shape that has a clean ringlet appearance rather than being frizzy, curls that appear more individualized, curls that are more closed in appearance, and/or curls that have an improved visual appearance of the hair color and/or highlights.

A “leave-in” composition or product refers to a composition such as a hair-treatment composition that is not rinsed and/or washed away with water or acceptable solvent after the application of the composition onto the keratin fiber, such as hair; instead, the composition is allowed to remain on the keratin fibers for a period of time as desired, such from 1 hour, 2 hours, 3 hours, 4 hours, up to 8 hours, overnight, or as long as needed, until next time of washing or rinsing the keratin fibers.

As used herein, the term “salts” refers to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

It is to be understood that, with regard to salts of carboxylic acids described herein, it is intended to encompass the use of a salt of a carboxylic acid as an ingredient added to a composition according to the disclosure, or to the salt of the carboxylic acid that forms when the carboxylic acid is used as an ingredient in a composition according to the disclosure.

As used herein, the term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “treat” (and its grammatical variations) refers to the application of the compositions of the present disclosure onto the surface of keratin materials, such as hair.

As used herein, a “bleach composition” or “bleaching composition” is a composition comprising one or more components that bleach or lift the color of the hair. Such components are known and may include, for example, peroxides, persulfates, perborates, percarbonates, peracids, silicates, or salts thereof.

As used herein, a “bleaching process” is a process for bleaching or lifting the color of the hair, and typically includes applying a bleaching composition to the hair and allowing the bleaching composition to remain on the hair until the degree of lift is achieved.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following Examples are intended to be non-limiting and explanatory in nature only. In the Examples, amounts are expressed in percentage by weight (wt %) of active materials, relative to the total weight of the composition, unless otherwise indicated.

### Example 1—Compositions

### 1.1 Treatment Compositions

Exemplary comparative compositions C1 and C2 and inventive composition 1A were prepared according to the formulations set forth in Table 1-1.

TABLE 1-1

| | Inventive | Comparative | |
|---|---|---|---|
| | 1A | C1 | C2 |
| XANTHAN GUM | 1 | 1 | |
| GLYCERIN | 1 | 1 | |
| CITRIC ACID | 2.5 | | 2.5 |
| CYCLODEXTRIN | 1.5 | | 1.5 |
| WATER | QS | QS | QS |

Composition C1 was prepared by mixing xanthan gum and glycerin in water, and adjusting the pH of the mixture to about 3.5-4 at 25° C. using 0.1 M sodium hydroxide (NaOH) solution. Composition C2 was prepared by diluting a premixture of citric acid and cyclodextrin in deionized water and adjusting the pH of the dilution to about 3.5-4 at 25° C. using 0.1 M NaOH solution. The premixture of citric acid (CA) and cyclodextrin (CD) was prepared by mixing citric acid, cyclodextrin, and deionized water (DI) in a mole ratio of CD:CA:DI at 1:10:50, corresponding to a weight ratio of CD:CA:DI at 29:48:23, and heating the mixture in a water bath at about 80° C. until CA and CD were dissolved in the DI water and the mixture formed into viscous liquid, leaving the mixture in the water bath for a period of about 2 hours, and then cooling down the mixture to room temperature (RT). Composition 1A was prepared by diluting the premixture of citric acid and cyclodextrin described above in deionized water and then adjusting the pH of the dilution to about 3.5-4 using 0.1M sodium hydroxide solution; next, adding xanthan gum and glycerin to the solution and mixing the combination using a vortex stirrer (IKA® EUROSTAR Power Control from IKA Werke) for about an hour to ensure that an homogeneous gel is obtained.

### 1.2 Exemplary Bleach Composition

An exemplary bleach composition was prepared according to the formulation set forth in Table 1-2.

TABLE 1-2

| | Bleach |
|---|---|
| | Powder |
| CYAMOPSIS TETRAGONOLOBA | 2.08 |
| (GUAR) GUM/CYAMOPSIS | |
| TETRAGONOLOBA GUM | |
| MAGNESIUM CARBONATE HYDROXIDE | 7.44 |
| DISODIUM EDTA | 0.99 |
| SODIUM SILICATE | 17.36 |
| POTASSIUM PERSULFATE | 49.61 |
| SODIUM METASILICATE | 2.48 |
| MAGNESIUM CARBONATE HYDROXIDE | 1.98 |
| SODIUM STEARATE | 9.92 |
| AMMONIUM PERSULFATE | 4.96 |
| PANTHENOL | 0.11 |
| ULTRAMARINES | 0.08 |
| SODIUM LAURYL SULFATE | 0.99 |
| MINERAL OIL/PARAFFINUM LIQUIDUM | 1.98 |

### 1.3 Exemplary 30 V Developer Composition

The exemplary 30V developer composition D set forth in Table 1-3 was prepared. The developer D has a pH at 25° C. of about 2.2±0.2.

TABLE 1-3

| | 30V Developer |
|---|---|
| | Composition D |
| TRIDECETH-2 CARBOXAMIDE MEA | 0.85 |
| TETRASODIUM ETIDRONATE | 0.2 |
| SODIUM SALICYLATE | 0.035 |
| GLYCERIN | 0.5 |
| CETEARYL ALCOHOL (and) | 2.85 |
| CETEARETH-25 | |
| HYDROGEN PEROXIDE | 18 |
| TETRASODIUM PYROPHOSPHATE | 0.04 |
| WATER | QS |

### Example 2—Evaluation Methods

The properties of hair were visually evaluated and/or evaluated by one or more instrumental assessments according to the following process or protocols.

### 2.1 Fiber Integrity Evaluation

Fiber integrity evaluation was carried out using a Miniature Tensile Tester (MTT 688 from Dia-Stron Ltd) and a Fibre Dimensional Analysis System (FDAS) 770. Fifty (50) fibers per hair sample were tested in a wet or dry state.

Each fiber cross-section area of the tested hair sample was measured using the FDAS 770 according to a double-ended method. After the fiber cross-section area was measured, tensile strength and elastic modulus of the fibers were measured using the MTT in dry and the wet states. For measurement in dry state, the hair swatch samples were let to equilibrate at 45% relative humidity (45% RH) at room temperature for about 24 hours and subsequently subjected to tensile testing using MTT with 2 grams of force at a traction rate of about 40 mm/min. For measurement in wet MTT, each hair swatch sample was soaked in deionized (DI) water for about two (2) hours, and subsequently subjected to tensile testing using MTT. Tensile measurement was performed while the hair fibers were wet, with 2 grams of force at a traction rate of about 40 mm/min. The measured results of the hair break stress and elastic modulus were statistically analyzed using UVWin software vision 4.2.5.0, where the statistical significance level was set at p<0.05.

Elastic modulus represents a measure of the hair's spring-like structure (elasticity). Elastic modulus provides fiber deformation information, so fiber with higher elastic modulus is more resistant to deform. Break stress represents the force/area needed to break the hair fiber. A higher break stress represents a stronger hair fiber. Overall, the higher the break stress and/or the elastic modulus, the better the fiber integrity.

### 2.2 Differential Scanning Calorimetry (DSC) Measurement

Differential Scanning calorimetry (DSC) measures the temperatures and heat flow associated with the thermal transition in a material. It is useful for investigating the structural characteristics of hair fibers. Keratin undergoes detectable transformations at various temperatures. Changes in these transformation temperatures can be used to estimate how a particular hair-treatment that may impact hair fibers. In this study, DSC was used to measure denaturation temperature (T_{d}). Denaturation temperature (T_{d}) has been used as a representation of the thermal stability of hair fibers, which is influenced, at least in part, by the cross-link density of the matrix (intermediate filament associated proteins, IFAP). Thermal stability (T_{d}) and its relationship in determining the thermal stability of hair fibers is established in the literature. Generally, the denaturation temperature of hair is higher if the hair strength/integrity is high and vice-versa.

The treated hair swatches were evaluated using DSC (DSC 2500 Discovery series from TA Instruments). To prepare hair samples prior to DSC analysis, 7-8 grams of each treated hair swatch was cut into particles with fine size (<0.5 mm). The hair particles were placed in a high-volume DSC pan chamber. About 50 μL of deionized water (DI water) was added into the pan and the hair particles were soaked in the water. The pan chamber was then sealed using a crimping dime. For each treated hair swatch samples, four pans were prepared (i.e. n=4).

The hair samples were equilibrated at room temperature for overnight. Approximately 24 hours later the hair samples were run on the DSC equipment starting from 40° C. and ramping by 10° C./min to 180° C.

T_{d} value was measured in degree Celsius, which represents the cross-linking density of the hair matrix. Trios' software was used to statistically analyze the denaturation temperature values measured by the DSC equipment. An increase in T_{d} indicates interaction of active and matrix hair protein.

### 2.3 Fiber Durability Testing (Cyclic Fatigue Tensile Testing, CFTT)

Cyclic fatigue testing is typically used to simulate the repeated tensile stress that occurs while combing and brushing. Cyclic fatigue data and survival probability, present both internal fiber flaws and external cuticle layer rupture. Cyclic fatigue testing provides a unique instrumental tool to demonstrate the progressive levels of impaired hair fibers. Fibers with relatively large flaws probably will break at lower numbers of cycles of applied stress than fibers with minor defects.

Fiber durability testing (cycles to break (CTB)) was carried out using a Cyclic Fatigue Tensile Tester (CFTT) 801 from Dia-Stron Ltd. The hair sample size for each swatch was about 50 fibers per sample. The fibers were 28 mm long. The experiment was carried out at about 23° C. and 45% relative humidity, and using a constant stress over a specified area.

### 2.4 Evaluation of Curl Definition

RUMBA is a polarization imaging system that takes successive images of a hair sample illuminated with infrared LED and under several polarization states, using a rotating polarizer. The images give a modulated intensity on each pixel. The modulation is caused by the birefringence of the hair fiber and contains the angle signature that can be extracted with the system's software to build an orientation image of the sample. The orientation data can be mapped onto a 2D image to visualize the whole sample. From this orientation data, a coefficient that relates to straightness of the hair can be defined—the straighter the hair fibers in the swatch, the closer to a single angle value the distribution of the orientation will be. The standard deviation of such a distribution is therefore inversely proportional to the perception of straightness. The same process is done for the alignment, where the alignment of a hair swatch is defined as the hair fibers being “locally straight.” Thus, a straightness coefficient is defined on a small scale of the 2D mapping of the orientation, which allows for a straightness coefficient to be obtained on every pixel of the image.

After the hair swatches were exposed to the condition of about 80% RH at 27° C. for about 24 hours and removed from the HEAVEN system, curl definitions of the hair swatches were analyzed using RUMBA (Orientation measurement for straightness & Alignment of hair fiber) from Bossa Nova Technologies, LLC and/or with photographic images. Each individual hair swatch was placed in the RUMBA and Rumba images were acquired for the hair swatches exposed to a condition of 80% relative humidity at 27° C. for 24 hours.

### Example 3—Methods for Treating Hair

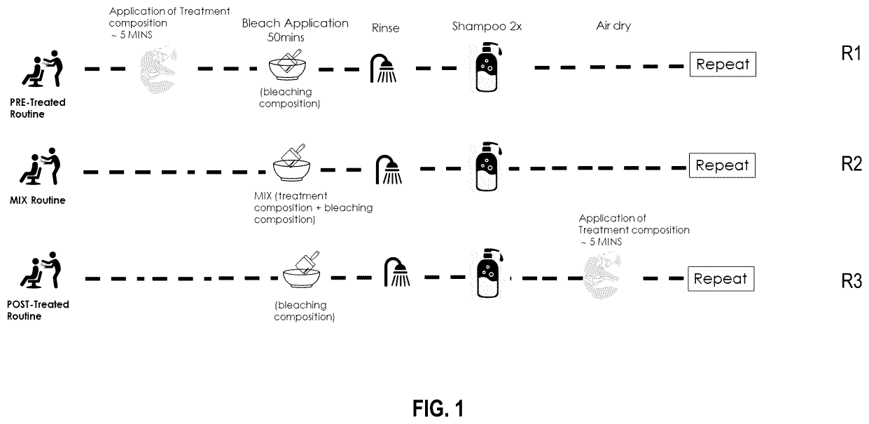

Hair was treated according to a general bleach process (or general bleach routine) and three tested bleach processes (or tested bleach routines). The three bleach routines are: pre-treatment bleach routine (R1, or pre-trt), mix-treatment bleach routine (R2, or mix-trt), and post-treatment bleach routine (R3, or post-trt). The three tested bleach routines are further illustrated in the schematic diagrams in FIG. 1.

As can be seen in FIG. 1, in the pre-treatment bleach routine (R1), the hair is pre-treated with a treatment composition according to the present disclosure or a comparative composition, such as composition 1A, C1, or C2, before the bleaching composition is applied. In the mix-treatment routine (R2), the hair was treated with a mixture of a bleaching composition and a treatment composition according to the present disclosure or a comparative composition. In the post-treatment bleach routine (R3), the hair is post-treated with a treatment composition according to the present disclosure or a comparative composition after the hair was treated with a bleaching composition.

The processes or protocols of these three bleach routines tested in the following Examples are detailed below. Commercially available shampoo, Redken Flash lift bleach powder, and Redken Pro-Oxide Cream 30 Vol Developer were used as examples and are not limiting. Each hair swatch used in the Examples was about 2 grams.

### 3-1. Protocol of General Bleach Process (Routine)

1) Mix Redken Flash Lift Bleach Powder and Redken Pro-Oxide Cream 30 Vol Developer in a ratio of 1:2 (bleach powder:developer) to prepare a bleaching composition (or bleaching mixture).
2) Pre-wash a hair swatch subjected to testing using shampoo at a ratio of about 0.4 gram of shampoo per gram of hair.
3) After washing the hair swatch, remove extra water from the hair using a towel and allow the hair to air dry.
4) Apply about 20 grams of the bleaching composition prepared in step 1) to the pre-washed and air-dried hair swatch at a ratio of about 10 gram of the bleaching composition per gram of hair, and evenly distribute the bleaching composition on the hair by combing through the hair using an applicator brush for about 1 minute per side and a total of about 2 minutes.
5) Hang the hair swatch treated with the bleaching composition in an environmental chamber having 45% of relative humidity at 30° C. for about 50 minutes.
6) After exposing to 45% relative humidity for about 50 minutes, rinse the hair swatch with water for about 2 minutes, and then wash the hair swatch twice using shampoo.
7) After washing the hair swatch, allow the hair swatch to air dry overnight.
8) Optionally, after allowing the hair swatch to air dry overnight, repeat steps 4-7 one or more times as desired, each repeat starting with the hair air dried overnight. For example, when the hair swatches are bleached twice, steps 4-7 are repeated twice.

In a modified general bleach routine, step 4) and step 5) are modified as below: after applying the bleaching composition to the pre-washed hair swatch in above step 4), the hair is wrapped with a foil and then the bleaching composition is distributed evenly on the hair by combing through the hair using an application brush for about 1 minute per side and a total of about 2 minutes. In step 5), the hair swatch is placed in an environmental chamber having 60% relative humidity at 30° C. for about 50 minutes.

### 3-2. Protocol of Pre-Treatment Bleach Process (Routine)

1) Mix Redken Flash Lift Bleach Powder and Redken Pro-Oxide Cream 30 Vol Developer in a ratio of 1:2 (bleach powder:developer) to prepare a bleaching composition (or bleaching mixture).
2) Pre-wash a hair swatch (2 grams) subjected to testing using shampoo at a ratio of about 0.4 gram of shampoo per gram of hair.
3) After washing the hair swatch, remove extra water from the hair using a towel and allow the hair to air dry.
4) Apply 0.4 grams of a testing treatment composition, e.g., composition 1A according to the present disclosure, or comparative composition C1 or C2, to the pre-washed and air-dried hair swatch.
5) Massage each hair swatch applied with the testing treatment composition for about 1 minute to distribute the testing treatment composition evenly on the hair, and leave the treatment composition on the hair for about 4 minutes, with a total of about 5 minutes of treatment.
6) Apply about 20 grams of the bleaching composition prepared in step 1) to the hair swatch while the testing treatment composition is left on the hair (10 grams of the bleaching mixture per gram of hair), and evenly distribute the bleaching composition on the hair by combing through the hair using an applicator brush for about 1 minute per side and a total of 2 minutes.
7) Hang the hair swatch treated with the bleaching composition in an environmental chamber having 45% of relative humidity at 30° C. for about 50 minutes.
8) After exposing to 45% RH for about 50 minutes, rinse the hair swatch with water for about 2 minutes, and then wash the hair swatch twice using DOP shampoo.
9) After washing the hair swatch, remove extra water from the hair and allow the hair swatch to air dry overnight.
10) Optionally, after the hair swatch is air dried overnight, repeat steps 4-9 one or more times as desired, each repeat starting with the hair air-dried overnight. For example, when the hair swatches are bleached twice, steps 4-9 are repeated twice.

In a modified pre-treatment bleach routine, step 6) and step 7) are modified as below: after applying the bleaching composition to the hair in above step 6), the hair is wrapped with a foil and then the bleaching composition is distributed evenly on the hair by combing through the hair using an application brush for about 1 minute per side and a total of about 2 minutes. In step 7), the hair swatch is placed in an environmental chamber having 60% relative humidity at 30° C. for about 50 minutes.

### 3-3 Protocol of Mix-Treatment Bleach Process (Routine)

1) Pre-wash a hair swatch (20 grams) subjected to testing using shampoo at a ratio of about 0.4 grams of shampoo per gram of hair.
2) After washing the hair swatch, remove extra water from the hair using a towel and allow the hair to air dry.
3) Mix Redken Flash Lift Bleach Powder and Redken Pro-Oxide Cream 30 Vol Developer in a ratio of 1:2 (bleach powder:developer) to prepare a bleaching composition (or bleaching mixture).
4) Separately add 0.4 gram of testing treatment compositions 1A, C1, and C2 into 20 g of the bleaching composition obtained in step 3) and then mix the mixture, followed with adjusting the pH of the mixture to 9.5-10 using monoethanolamine to obtain a treatment and bleaching mixture.
5) Apply the homogenized treatment and bleaching mixture to the pre-washed hair swatch, and evenly distribute the treatment and bleaching mixture on the hair by combing through the hair using an applicator brush for about 1 minute per side and a total of 2 minutes.
6) Place the hair swatch treated with the treatment and bleaching mixture in an environmental chamber having 45% of relative humidity at 30° C. for about 50 minutes.
7) After exposing to 45% relative humidity for about 50 minutes, rinse the hair swatch with water for about 2 minutes, and then wash the hair swatch twice using DOP shampoo.
8) After washing the hair swatch, remove extra water from the hair, and then optionally scrunch the hair five times (5×) to aid curl definition and add style. After then, allow the hair swatch to air dry overnight.
9) Optionally, after air drying the hair swatch overnight, repeat steps 4-10 one or more times as desired, each repeat starting with the hair air-dried overnight. For example, when the hair swatches are treated and bleached twice, steps 5)-11) are repeated twice.

### 3-4 Protocol of Post-Treatment Bleach Process (Routine)

1) Mix Redken Flash Lift Bleach Powder and Redken Pro-Oxide Cream 30 Vol Developer in a ratio of 1:2 (bleach powder:developer) to prepare a bleaching composition (or bleaching mixture).
2) Pre-wash a hair swatch (2 grams) subjected to testing using shampoo at a ratio of about 0.4 grams of shampoo per gram of hair.
3) After washing the hair swatch, remove extra water from the hair using a towel and allow the hair to air dry.
4) Apply about 20 grams of the bleaching composition prepared in step 1) to the pre-washed and air-dried hair swatch at a ratio of about 10 gram of the bleaching composition per gram of hair, and evenly distribute the bleaching composition on the hair by combing through the hair using an applicator brush for about 1 minute per side and a total of 2 minutes.
5) Place the hair swatch treated with the bleaching composition in an environmental chamber having 45% of relative humidity at 30° C. for about 50 minutes.
6) After exposing to 45% relative humidity for about 50 minutes, rinse the hair swatch with water for about 2 minutes, and then wash the hair swatch twice using DOP shampoo.
7) After the hair swatches are washed twice using DOP shampoo, apply 0.4 grams of a treatment composition, e.g., composition 1A according to the present disclosure or comparative composition C1 or C2, to the hair swatch, and massage the hair swatch applied with the treatment composition for about 1 minute, followed with scrunching the hair five times (5×) to aid curl definition and add style. After then, allow the hair swatch to air dry overnight.
8) Optionally, repeat step 4)-7) as desired, each repeat starting with the hair air-dried overnight. For example, when the hair swatches are bleached twice, steps 4)-7) are repeated twice.

### Example 4—Evaluation of Performance in Pre-Treatment Bleach Routine

The performance of composition 1A were evaluated on Caucasian curly hair swatches subjected to bleach in comparison with the performance of comparative compositions C1 and C2. Hair swatches were respectively treated with compositions 1A, C1, or C2 and bleached twice following the protocols for pre-treatment bleach routine, mix-treatment bleach routine, and post-treatment bleach routine. Untreated hair swatches and hair swatches that were treated twice with the bleaching composition alone following the general bleach routine protocol used as controls. Each hair swatch was about 2.0 grams.

After treating the hair swatches according to different routines as described above, the properties of the treated hair swatches were evaluated visually and/or using instruments according to the evaluation methods described above. The evaluations and results are detailed below.

### 4-1 Visual Appearance of Hair Swatches Treated Via Different Bleach Routines

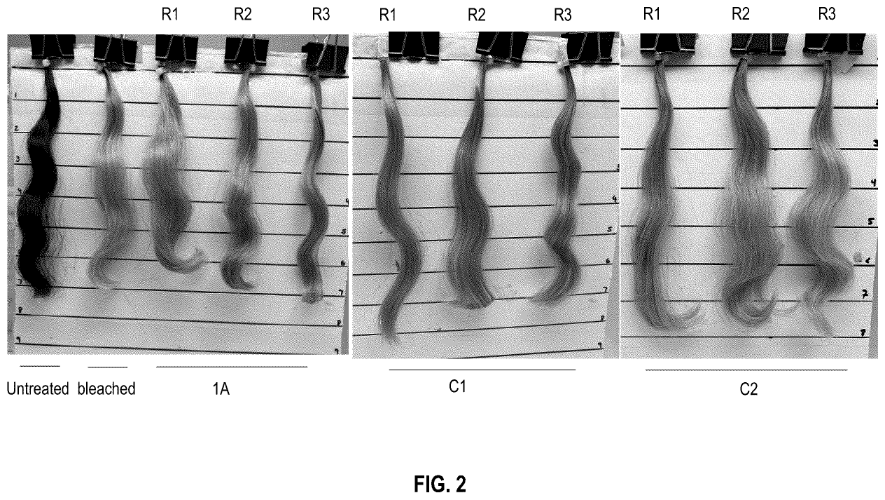

Photographs of the untreated hair swatch, a hair swatch that was treated with the bleaching composition twice (2×) alone, and the hair swatches respectively treated twice following pre-treatment bleach routine, mix-treatment bleach routine, and post-treatment bleach routine, where the hair swatches were respectively treated with compositions 1A, C1, and C2 before, during, or after the hair was treated with the bleaching composition, were taken at the end of the whole process and are shown in FIG. 2.

As can be seen in FIG. 2, the hair bleached twice using the bleaching composition alone had curls that was noticeably less defined compared to the untreated hair. In contrast, the hair swatches that were treated via the pre-treatment and post-treatment routines, where the hair was treated with composition 1A before or after the hair swatches were treated with the bleaching composition, had noticeably better curls, less frizzy, and better discipline, compared to the hair bleached alone and the hair treated via a mix-treatment bleach routine, where composition 1A and the bleaching composition were applied to the hair together in a mixture.

Further, as can be seen in FIG. 2, the hair swatches respectively treated with comparative compositions C1 and C2 via pre-treatment, mixt-treatment, or post-treatment bleach routines, where compositions C1 and C2 were applied to the hair before, during, or after the hair was treated with the bleaching composition, had noticeable curl elongation with less defined curls and/or the hair was frizzier compared to the hair treated with composition 1A before or after the hair was bleached. In addition, in all mixt-treatment, pre-treatment, and post-treatment routines, the hair swatches that were treated with composition 1A and C2 showed better color lifting than the hair swatches treated with composition C1.

The results demonstrate that the combination of a carboxylic acid such as citric acid, cyclodextrin, a polysaccharide such as xanthan gum, and a polyol such as glycerin in water, when being used to treat hair before or after bleaching the hair, provided synergistic benefits such as maintaining and/or preventing elongation of curly hair, and/or maintaining and/or improving curl definition and hold, to the hair subjected to bleach.

### 4-2 Fiber Integrity of Hair Treated Via Pre-treatment Bleach Routine

The fiber integrities of the hair swatches treated following the pre-treatment bleach routine twice, where composition 1A and compositions C1 and C2 were respectively applied to a separate hair swatch before the application of the bleaching composition, were measured using a Miniature Tensile Tester (MTT 688, from Dia-Stron Ltd) and statistically analyzed according to the process described above, in comparison with the untreated hair and bleached only hair (bleached 2×).

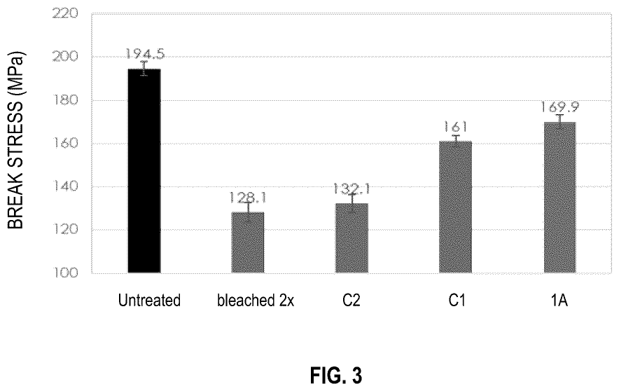

The results of the break stress of the treated and bleached hair swatches are illustrated in FIG. 3. As shown in FIG. 3, after the hair was bleached twice alone, the break stress of the hair significantly decreased, indicating that the hair was damaged due to the chemical treatment of the bleaching composition. Pre-treating the hair using composition C2 did not significantly affect the break stress of the hair, indicating that composition C2 did not significantly reduce the hair damage. In contrast, hair swatches that were pre-treated with composition 1A and composition C1, respectively, before the application of the bleaching composition showed significantly improved break stress, compared to the bleached alone control hair, indicating that the pre-treatment using compositions A1 and C2, respectively, restored the integrity of the hair fiber and reduced the damage to the hair due to the chemical treatment of the bleaching composition. Further, as can be seen in FIG. 3, the bleached hair that was pre-treated with composition 1A had higher break stress than that of the bleached hair that was pre-treated with composition C1. As such, composition 1A outperformed than composition C1.

The results demonstrate the benefits of restoring or improving fiber integrity and thus reducing damage to the hair provided by the synergistic combination of the components according to the present disclosure, as included in composition 1A.

### 4-3 Fiber Durability of Hair Treated Via Pre-treatment Bleach Routine

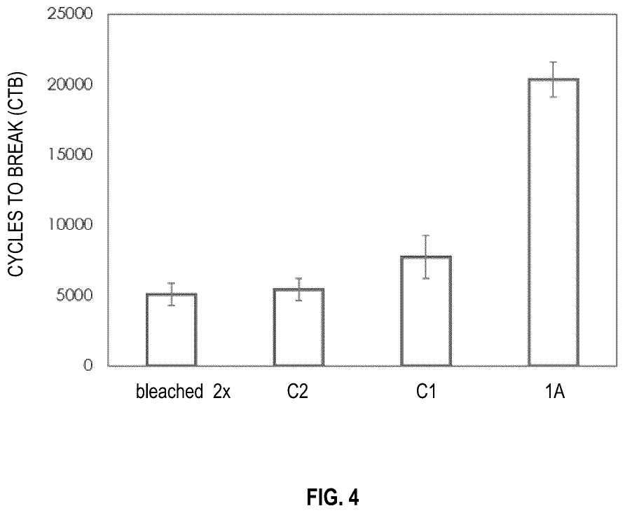

The fiber integrities of the hair swatches treated following the pre-treatment bleach routine twice, where composition 1A and compositions C1 and C2 were respectively applied to a separate hair swatch before the application of the bleaching composition, were evaluated by cyclic fatigue testing according to the process described above. Obtained cyclic fatigue results are shown in FIG. 4.

As shown in FIG. 4, the cycles needed to break the hair that was pre-treated with composition 1A before the application of the bleaching composition were significantly more than the cycles needed to break the hair bleached with the bleaching composition alone or the hair pre-treated with compositions C1 and C2 before the application of the bleaching composition. The results demonstrated that the synergistic combination of the components included in composition 1A significantly enhanced fiber durability to the hair subjected to bleach provided.

### 4-4 Fiber Integrity of Hair Treated With Composition 1A Via Different Routines

The fiber integrities of the hair swatches respectively treated twice following the protocols of the pre-treatment bleach routine, mix-treatment bleach routine, and post-treatment bleach routine described above, where composition 1A was applied to a separate hair swatch before, during, or after the hair swatch was applied with the bleaching composition, were evaluated using the Miniature Tensile Tester (MTT 688, from Dia-Stron Ltd) when the hair was in wet state (and statistically analyzed according to the process described above, in comparison with the untreated hair and bleached only hair (bleached 2×).

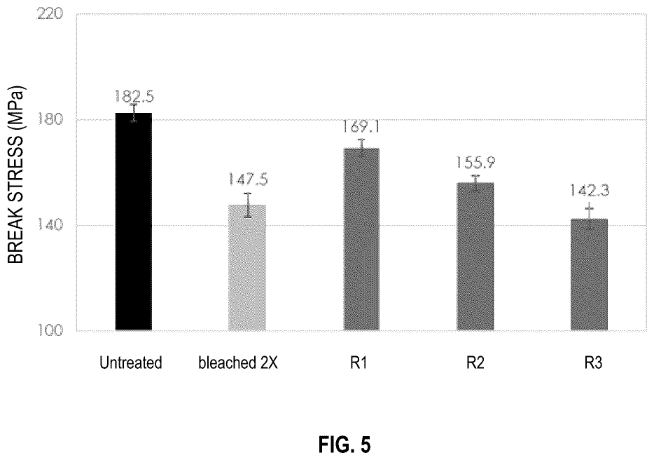

The results of the break stress of the hair swatches treated with composition 1A in different bleach routines compared to untreated and bleached 2× hair swatches are illustrated in FIG. 5. As shown in FIG. 5, after the hair was bleached twice using the bleaching composition alone, the break stress of the hair significantly decreased, indicating that the hair was damaged due to the chemical treatment of the bleaching composition. However, when the hair was pre-treated with composition 1A before the hair was treated with the bleaching composition, the break stress of the hair was significantly improved, compared to the hair bleached alone and the hair treated with composition 1A during or after the hair was applied with the bleaching composition. The 1A pre-treatment routine treated hair had significantly higher break stress compared to 2× bleach, mix-treatment, and post-treatment. The results showed that among the 3 different routines, pre-treatment showed the best damage prevention.

### 4-5 Fiber Durability of Hair Treated Via Different Routines

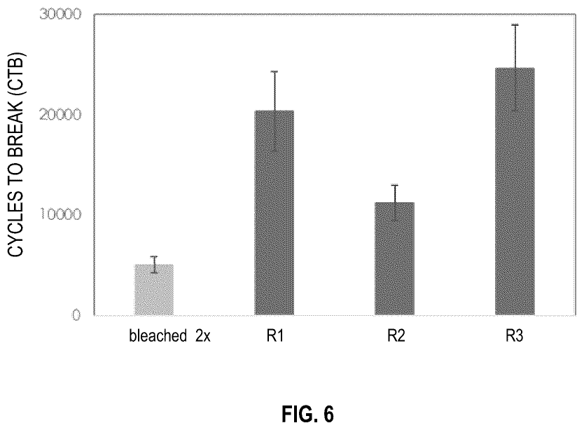

The fiber durability (cycles to break (CTB)) of the hair swatches respectively treated twice according to the pre-treatment bleach routine, mix-treatment bleach routine, and post-treatment bleach routine described above, where composition 1A was applied to a separate hair swatch before, during, or after the hair swatch was applied with the bleaching composition, were evaluated by cyclic fatigue testing according to the process described above. Obtained cyclic fatigue results are shown in FIG. 6.

As shown in FIG. 6, the cycles needed to break the hair swatches treated with composition 1A before, during, or after the application of the bleaching composition were significantly more than the cycles needed to break the hair treated with the bleaching composition along. As such, the treatment using composition 1A in all of the pre-treatment, mix-treatment, and post-treatment bleach routines improved the fiber durability of the bleached hair. The results further shows that the hair treated via the pre-treatment and post-treatment routine had significantly higher cycles to break value than the hair treated via the mix-treatment routine.

### 4-6 Denaturation Temperature of Hair Treated Via Different Routines

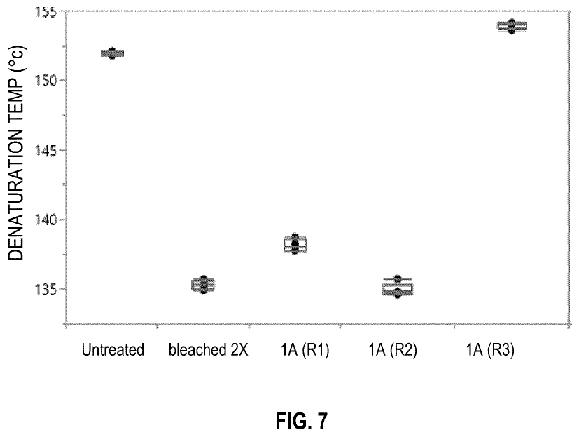

The denaturation temperatures of the hair swatches treated with composition 1A in different bleach routines, where composition 1A was applied to the hair before, during, or after the hair was applied with the bleaching composition, were evaluated using DSC according to the process described above, in comparison with the untreated hair swatch and the hair swatch bleached twice with the bleaching composition alone. The results are seen in FIG. 7.

As shown in FIG. 7, compared to the untreated hair, the hair treated with the bleaching composition twice alone (bleached 2×) had a significant lower denaturation temperature than that of the untreated hair, indicating that the hair was damaged during the treatment with the bleaching composition. However, hair treated via the pre-treatment bleach routine and the post-treatment bleach routine with composition 1A had increased denaturation temperature compared to the bleached 2× hair, and the increase was significant when the hair was treated via the post-treatment bleach routine.

The results demonstrate that pre-treatment and post-treatment of the hair using composition 1A significantly enhances the hair fiber's thermal integrity.

### 4-7 Curl Definition of Hair Treated Via Different Routines

The curl definition of the hair swatches treated according to the pre-treatment, mix-treatment, and post-treatment bleach routines as described above, where composition 1A was used as the treatment composition and applied to the hair before, during, or after the bleaching composition, was evaluated using a RUMBA system as described above.

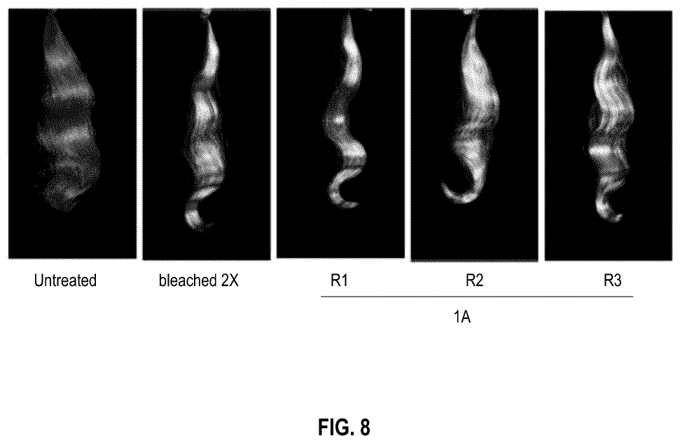

The 2D images obtained with the RUMBA system for the treated hair swatches are shown in FIG. 8. As can be seen in FIG. 8, the hair treated with using 1A in all pre-treatment, mix-treatment, and post-treatment routines did not have curl elongation and was less frizzy compared to the hair bleached alone. Further, the hair swatches treated and bleached via pre-treatment and post-treatment bleach routines showed better curl definition, compared to the hair bleached alone (bleached 2×) and the hair treated via the mix-treatment routine. The results demonstrated that composition 1A used in any of the pre-treatment or post-treatment bleach routines disclosed herein provided prevented curl elongation and provided better curl definition and manageability to the curly hair.

### 4-8 Summary of the Study Results

The performance of composition 1A in pre-treatment bleach routine, mix-treatment bleach routine, and post-treatment bleach routine, in comparison with comparative composition C1 and C2, are summarized below in Table 2.

TABLE 2

| | Wet MTT | | Dry MTT | | | |
|---|---|---|---|---|---|---|
| | (Covalent bonds) | | (Fiber quality) | | CFTT | |
| | Elastic | Break | Elastic | | Cycles | |
| Bleach | Modulus | Stress | Modulus | Tough- | to Break | DSC |
| Treatment | (EM) | (BS) | (EM) | ness | (CTB) | Td |
| Pre-trt-1A | ✓ | ✓ | ✓ | ✓ | ✓ | ✓ |
| Pre-trt-C2 | | | | | | ✓ |
| Pre-trt-C1 | ✓ | ✓ | ✓ | | | |
| Post-trt-1A | N/A | N/A | ✓ | ✓ | ✓ | ✓ |
| Post-trt-C2 | N/A | N/A | | | | ✓ |
| Post-trt-C1 | N/A | N/A | | | | |
| Mix-trt-1A | | | | | ✓ | |
| Mix-trt-C2 | | | | | | |
| Mix-trt-C1 | ✓ | ✓ | ✓ | | | |

In sum, composition 1A, when it was applied to the hair before or after the hair was applied with the bleaching composition, surprisingly provided overall improvements in fiber quality, covalent bonds, hair durability, and hair strength, compared to when composition 1A was applied to the hair in a mixture with the bleaching composition. Further, the results demonstrate that the benefits were due to the synergistic combination of components included in composition 1A.

### Example 5—Performance of Composition 1A on African American Hair

The performance of composition 1A was tested and evaluated on African American hair swatches having tight curls following the modified pre-treatment bleach routine described above, compared to an untreated swatch and a hair swatch treated with the bleaching composition alone following the modified general bleach routine protocol described above. Hair swatches were bleached once and twice respectively. Each hair swatch used in the testing was about 2.0 grams.

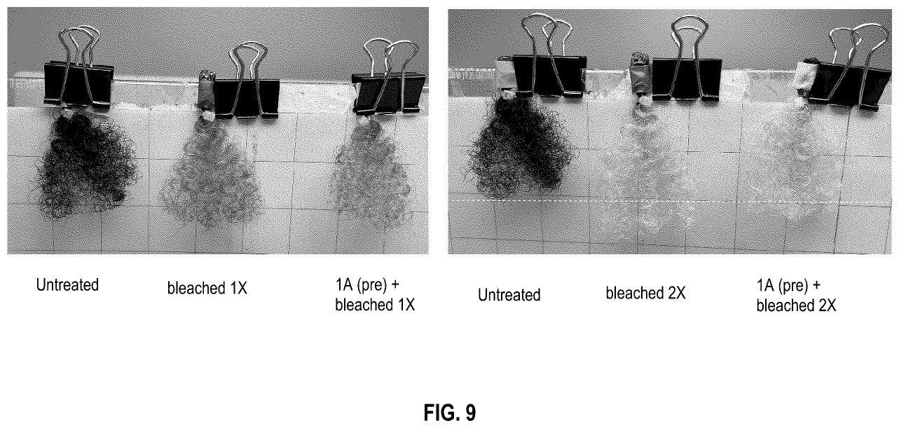

FIG. 9 shows the appearance of the hair swatches treated with the bleaching composition alone once (bleached 1×) and twice (bleached 2×), and the hair swatches treated according to the pre-treatment bleach routine once (1A(pre)+bleached 1×) and twice (1A(pre)+bleached 2×), where the hair was pre-treated with composition 1A before the application of the bleaching composition, compared with the appearance of an untreated hair swatch.

As can be seen in FIG. 9, the hair swatches that were bleached twice show better color lifting than the hair swatches that were bleached once. However, after the hair swatch was treated with the bleaching composition alone twice, the hair swatch showed noticeable elongation. In contrast, the hair swatch that was treated according to the pre-treatment bleach routine, where the hair was treated with composition 1A before the application of the bleaching composition, showed significantly reduced elongation with maintained or improved curl definition. The color lifting effect was not affected by the pretreatment with composition 1A.

### Example 6—Performance of Composition 1A on Different Types of Curly Hair

The performance of composition 1A is compared on two different hair swatches, Caucasian curly hair swatches having loose curls and African American hair swatches having tight curls. Natural Caucasian curly hair generally has lower cycles to break value and is less durable than natural African-American hair.

Both type of hair swatches were treated twice according to the pre-treatment bleach routine describes above, where the hair was treated with composition 1A before the application of the bleaching composition, in comparison with hair swatches treated with the bleaching composition alone twice according to the general bleach routine.

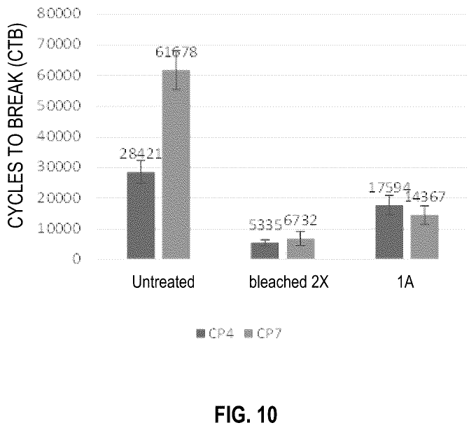

The treated hair swatches were evaluated using fiber durability testing according to the evaluate method described above. Obtained cyclic fatigue (cycles to beak (CTB)) results are shown in FIG. 10.

As shown in FIG. 10, after the treatment using the bleaching composition alone, the cycles needed to break the hair swatches of both types were significantly reduced, indicating that both types of hair were damaged due to the chemical treatment using the bleaching composition. However, when the hair swatches of both types were treated with composition 1A before being treated with the bleaching composition, the cycles needed to break the hair both significantly improved, indicating that the hair was more durable and less damaged compared to the hair that was bleached only.

The results demonstrate that composition 1A provided benefits of improving the hair integrity and reducing damage to the hair regardless the curly level of hair, at least when composition 1A was used in a pre-treatment bleach routine.

The above Examples demonstrate that the synergistic combination of components according to the disclosure surprisingly prevented and/or reduced curl elongation, and restored and/or improved hair integrity to hair subjected to bleach. The Examples further demonstrate that the synergistic combination generally performed better when being used to treat the hair before or after the hair was applied with a bleaching composition.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1-29. (canceled)

30. A method for treating hair comprising applying to the hair a treatment composition comprising:
(a) at least one carboxylic acid or salt thereof;
(b) at least one cyclodextrin or derivative thereof;
(c) at least one polysaccharide thickening agent;
(d) at least one polyol; and
(e) water;
wherein the composition has a pH of less than 7; and
wherein the treatment composition is applied to the hair before, during, or after a bleaching process.

31. The method according to claim 30, wherein the treatment composition is applied to the hair before a bleaching process, and wherein the treatment composition is left on the hair for a period of time before a bleaching composition is applied to the hair.

32. The method according to claim 31, wherein the treatment composition is left on the hair for a period of time ranging from about 1 minute to about 30 minutes before the bleaching composition is applied to the hair.

33. The method according to claim 31, wherein the treatment composition is not rinsed from the hair before the bleaching composition is applied to the hair.

34. The method according to claim 30, wherein the treatment composition is applied to the hair during the bleaching process:
(i) before a bleaching composition is applied to the hair, wherein the treatment composition is left on the hair when the bleaching composition is applied to the hair;
(ii) after a bleaching composition is applied to the hair, wherein the bleaching composition is left on the hair when the treatment composition is applied to the hair; and/or
(iii) as a mixture of treatment composition and bleaching composition.

35. The method according to claim 30, wherein the treatment composition is applied to the hair after a bleaching process, and is applied to the hair after a bleaching composition is rinsed from the hair.

36. The method according to claim 35, wherein the treatment composition is left on the hair for a period of time ranging from about 3 minutes to about 10 minutes.

37. The method according to claim 30, wherein the treatment composition has a mole ratio of (a) carboxylic acids and salts thereof to (b) cyclodextrins and derivatives thereof ranging from about 0.5:1 to about 25:3.

38. The method according to claim 30, wherein the treatment composition has a weight ratio of the total amount of (a) carboxylic acids and salts thereof to (b) cyclodextrins and derivatives thereof ranging from about 1:3 to about 6:1.

39. The method according to claim 30, wherein the at least one carboxylic acid, salts thereof, or mixtures of two or more thereof is chosen from citric acid, lactic acid, malic acid, maleic acid, tartaric acid, glycolic acid, phytic acid, salts thereof, or mixtures of two or more thereof.

40. The method according to claim 30, wherein the total amount of carboxylic acids and salts thereof ranges from about 1% to about 20% by weight, relative to the total weight of the treatment composition.

41. The method according to claim 30, wherein the at least one cyclodextrin or derivative thereof is chosen from compounds of formula: 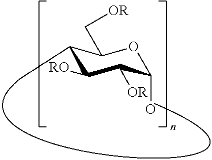
wherein:
R is chosen from H, CH₃, or a hydroxypropyl group; and
n is an integer ranging from 6 to 8.

42. The method according to claim 30, wherein the at least one cyclodextrin or derivative thereof is chosen from α-cyclodextrin, β-cyclodextrin, γ-cyclodextrin, methyl derivatives of α-cyclodextrin, methyl derivatives of β-cyclodextrin, methyl derivatives of γ-cyclodextrin, hydroxypropyl derivatives of α-cyclodextrin, hydroxypropyl derivatives of β-cyclodextrin, hydroxypropyl derivatives of γ-cyclodextrin, or mixtures of two or more thereof.

43. The method according to claim 30, wherein the total amount of cyclodextrins and derivatives thereof ranges from about 0.01% to about 20% by weight, relative to the total weight of the composition.

44. The method according to claim 30, wherein the at least one polysaccharide thickening agent is chosen from gum arabic, tragacanth gum, karaya gum, guar gum, gellan gum, tara gum, locust bean gum, tamarind gum, xanthan gum, dehydroxanthan gum, Seneca gum, sclerotium gum, gellan gum, or mixtures of two or more thereof.

45. The method according to claim 30, wherein the total amount of polysaccharide thickening agents ranges from 0.05% to about 5% by weight, relative to the total weight of the composition.

46. The method according to claim 30, wherein the total amount of polyols is at least 0.5% by weight, relative to the total weight of the composition.

47. A method for treating hair comprising applying to the hair a treatment composition comprising:
(a) from about 1% to about 5% of citric acid and/or a salt thereof;
(b) from about 1% to about 5% of β-cyclodextrin;
(c) at least one polysaccharide gum;
(d) at least one polyol; and
(e) water;
wherein the composition has a pH of less than 7; and
wherein the treatment composition is applied to the hair before, during, or after a bleaching process.

48. The method according to claim 47, wherein the treatment composition has a mole ratio of (a) citric acid and salts thereof to (b) β-cyclodextrin ranging from about 0.5:1 to about 25:3.

49. The method according to claim 47, wherein the pH of the treatment composition ranges from about 2 to about 6.
